(19) United States
(12) Patent Application Publication
(10) Pub. No.: US 2002/0165429 A1
(43) Pub. Date: Nov. 7, 2002

(54) Clitoral sensitizing arrangements

(75) Inventor: Ronald J. Thompson, Ft. Thomas, KY (US)

Correspondence Address: Donald N. Halgren, 35 Central Street, Manchester, MA, 01944-1311, US

(73) Assignee: 40 J's LLC

(21) Appl. No.: 09/736,973

(22) Filed: Dec. 14, 2000

Related U.S. Application Data
(63) Continuation-in-part of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61F 5/00

(52) U.S. Cl. 600/38

(57) ABSTRACT

This invention relates to an arrangement for the treatment of clitoral dysfunction of a female. Such clitoral dysfunction may be described as an excessively long arousal time from initiation of foreplay to complete clitoral erection, a decreased intensity of a woman's orgasm and a lack of multiple orgasms. The treatment for these clitoral dysfunctionalities include augmentation of testosterone for the female to supplement low testosterone levels and to improve the libido of the female and the treatment also includes a subsequent or concurrent application of a compound of menthol and L-Arginine applied to the clitoris of the female.

### BACKGROUND OF THE INVENTION

1. Field of the Invention

This invention relates to a method to increase the physiological actions of a topically applied clitoral compound by prior or concurrent administration of an oral or transdermal agent to increase central and peripheral female libido, and is a continuation-in-part application of my co-pending U.S. patent application Ser. No. 09/520,110 which is a continuation-in-part application of my co-pending application Ser. No. 09/468,959 which is a continuation-in part application of my co-pending application Ser. No. 09/414,250, which is a continuation-in-part application of my co-pending application Ser. No. 09/340,227, all of which are herein incorporated by reference, in their entirety.

2. Prior Art

Clitoral arousal and responsiveness are the primary factors in sexual enjoyment for females. Decreased clitoral sensitivity, and responsiveness are related to normal aging, relative or absolute estrogen and testosterone deficiency, (either as a consequence of medicines or aging), and by a host of vascular conditions such as diabetes and hypertension. Multiple laboratory and clinical research endeavors have been directed primarily toward male erectile dysfunction (ED), yielding not only an understanding of the erection physiology, but also medications to treat ED, such as VIAGRA®, a prescription medication marketed for that problem, by Pfizer, Inc. Very little research has been initiated to understand or address female physiological sexual unresponsiveness. However, since the penis and the clitoris are analogous anatomical structures, the basic cellular and physiological knowledge about male penile erections translates to functions of the clitoris.

Female clitoral dysfunction is extremely difficult to document and quantify. A number of modalities, such as Doppler blood flow, precise temperature measurements, and actual imaging measurements, have been employed to attempt to define clitoral erection have been reported in the literature-all with results unsatisfactory for meaningful research. Estimates that 15 million U.S. men suffer from erectile dysfunction have been reported in the literature. A recent article in the Feb. 10, 1999, issue of the Journal of the American Medical Association suggests that female erectile dysfunction occurs probably at twice the rate of male ED, therefore affecting 30 million women.

My co-pending patent application Ser. No. 09/469,959, filed Dec. 22, 1999, teaches the use of a topical clitoral sensitizing compound of menthol and L-arginine. The menthol component has two functions. First, the menthol causes a reflex vaginal lubrication, the first component of female sexual arousal. This response is mediated through the specific thermoreceptors and noiceptors in the mucous membrane of the vestibular tissue. In addition, the menthol acts as a vehicle to allow and facilitate the clitoral absorption of L-arginine, because of its extremely lipophilic nature. The L-arginine excess in the corpus cavenosa of the clitoris induces the nitric oxide synthase enzyme to produce nitric oxide. The nitric oxide causes the active dilation of the corpus cavenosa to engorge with blood to accomplish a clitoral erection, the second component of female arousal (the first was vaginal lubrication). Women can achieve orgasm only from a maximally aroused clitoris—a clitoral erection, analogous to a penile erection in men.

Female libido can best be described as a woman's desire or interest in having a sexual experience, satisfied preferably by an organism. A number of complex factors contribute to, or reduce, the immediacy or intensity of interest and desire. Two interrelated factors, physiological romance and hormonal status, modulate the intensity and immediacy of a woman's libido. In 1959, Waxenberg, et.al., postulated that a woman's testosterone level dictated her libido in “The Role of Hormones in Human Behavior I: Changes in Female Sexuality after Adrenalectomy” (Journal of Clinical Endocrinology, 19:193,1959). These findings were confirmed by Helen Singer Kaplan in the following medical journal and textbooks: “Hypoactive Sexual Desire.” Journal of Sex and Marital Therapy, 3(1):3-9, 1977; Disorders of Sexual Desire, New York: Brunner and Mazel, 1979; and The Evaluation of Sexual Disorders: Psychiatric and Medical Aspects, New York: Brunner and Mazel, 1983. In her 1993 article, “The Female Androgen Deficiency Syndrome,” (Journal of Sex and Marital Therapy, 19(1), 1993), Dr. Singer details the effects of testosterone deficiency on the sexual functioning of women. She outlines four points which support the notion that testosterone is a key hormone to the restoration of libido in women:

1. Normal females produce testosterone. The ovaries as well as the adrenal glands of normal women synthesize and secrete bio-active androgens.

2. There are testosterone receptors in female brains. Recent biomolecular studies have shown that certain neurons of both male and female brains are equipped with estrogen and testosterone receptors. These are concentrated in the areas that are involved with sex and emotions. This research has demonstrated that the sex-regulating centers of male and female brains are designed to react to the molecules of testosterone that are contained in the surrounding fluids. These fascinating new findings have provided a biological foundation for the concept that testosterone is involved in the modulation of the sexual motivation of both genders. 1. Androgen deficiency is associated with a loss of libido in females. The effects of androgen depletion in women were first documented in 1959, when Waxenberg and his colleagues astutely observed that women who had been treated for advanced breast cancer with the surgical ablation of their ovaries, adrenals, and/or hypophyses, and were thus deprived of all endrogenous sources of androgens, lost their libido and ability to respond to sexual stimulation. 2 Testosterone restores libido in androgen-deficient women. Albeit that no double-blind studies have been conducted so far, the reports of numerous investigators and clinicians who have found that testosterone replacement improves the symptoms of sexual inadequacy and restores libido to postmenopausal androgen-deficient women are impressive.

In the same article, Dr. Kaplan observed that the loss of testosterone within androgen-deficient female patients not only caused lack of libido, but also profoundly decreased the ability to have an orgasm, even with excessive clitoral stimulation. Orgasms in these patients, when achieved, were described as genitally localized and of only minor intensity. Both problems, libido, and, more importantly, the ability to become aroused and achieve a satisfactory orgasm, were resolved by treatment with testosterone injections. Testosterone is essential for an adequate libido and for the ability to achieve meaningful orgasms.

Testosterone is normal in females. Such testosterone is produced in three different sites: 25% from the ovaries, 25% from the adrenal gland, and 50% from adipose cell conversion of the substrate androstenedione to testosterone. Testosterone circulates in the blood in three states: 80% is tightly bound to a sex-binding globulin (a protein); 19% is loosely bound to albumin; and only 1% circulates unbound. The unbound testosterone is the only active and available fraction of the total circulating testosterone. At the cellular level, the testosterone will bind to a cell with a testosterone receptor, enter the cell, and be converted to dihydro testosterone, the only active androgen that evokes the androgen effect in that specific cell/organ. Target cells for testosterone include the brain, genitals, muscle cells, adipose cells, sebaceous cells, and hair follicles.

The major recognized causes of decreased testosterone effect in females are typically: (A) The normal aging process, as cited in Palmene, E. (ed.). Normal Aging. Durham, N.C.: Duke University, 1974; Palmene, E. (ed.). Normal Aging II. Duke Univsersity. 1980; and Morales, A. J. et. al. “Effects of Replacement Dose of DHEA in Men and Women of Advancing Age.” Clinical Endocinal Metabolism. 78:1360, 1994; (B) The long term use of Oral contraceptives as identified in Seagraves, T. R. “Hormones and Libido.” In Sexual Desire Disorders. Nw York: Guilford, 1988; (C) The long-term use of contraceptives: progesterone injections or implants as cited in World Health Organization, “Contraceptive Efficacy and Side Effects.” Contraception. 34:223, 1986, (A multi-centered phase III comparative clinical trial of depot-medroxyprogesterone acetate given in three monthly doses of 100 mg. or 150 mg.); and (D) Adrenalectomy or prolonged steroid use, as cited in Kaplan, H. S. “The Female Androgen Deficiency Syndrome.” Journal of sex and Marital Therapy. 19(1), 1993.

Therefore, it is an object of the present invention to provide a treatment arrangement for a woman's clitoral dysfunction whereby arousal time from the initiation of foreplay to complete clitoral erection is decreased by utilization of the present invention.

It is therefore a further object of the present invention to provide a treatment arrangement for a woman's clitoral dysfunction, whereby a woman may experience an increased intensity of her orgasm by utilization of the present invention.

It is therefore a still further object of the present invention, to provide a treatment arrangement for a woman's clitoral dysfunction whereby a woman may enjoy an increase in the number of multiple orgasms by utilization of the present invention.

### DESCRIPTION OF THE PREFERRED EMBODIMENTS OF THE INVENTION

The present invention includes methods to increase libido by augmenting reduced testosterone levels to synergistically support menthol and L-arginine application in testosterone deficient females by: 1. Testosterone ethenate (for example, by injection of typically about 5-10 mg daily); 2. S-Methyl-tertosterone (for example, sublingual application of typically 5-10 mg daily); and 3. Testosterone (for example-androgel, typically ½ the strength of men's application, or about 4-6 mg daily as transdermally applied). Alternatively, by the application to the female patient of “testosterone precursors” which precursors are converted to testosterone. Examples of such precursors are: 1. DHEA (Dehydroepiandrosterone) and 2. DHEA-S (Dehydroepiandrostrone-S).

Displacement of testosterone from the circulating loosely-bound testosterone (therefore elevating the free testosterone circulating) are also effected by herbal treatments of: 1. Angelica sinensis; 2. Withania somniferum and 3. Tarnera diffusa.

“Central libido” is the term applied to the desire or interest in having a sexual experience that is initiated or responded to by the brain. Testosterone acts on the testosterone receptors in the brain to adjust the libido, or desire, just as a thermostat adjusts the heat generated from a furnace. Recently, researchers have documented similar testosterone receptors in the rat penis. In the Journal of Urology Supplement, Sato et. al. state that “the paraventricular nucleus is known as an important brain area which mediates penile erection, and the nitric oxide synthase activity in the paraventricular nucleus is regulated by testosterone.” (163(4), 381). Sato concluded from his research that “Nitric oxide activity in the paraventricilar nucleus decreases with aging and is restored by testosterone replacement.” These finding suggest that “central libido” can be increased with an increased testosterone presence and that the method of action of the testosterone is to increase the activity of the nitric oxide synthase system.

“Peripheral libido” postulates that an increased testosterone milieu increases the activity of the nitric oxide synthase pathway in the peripheral organs, the clitoris, and the penis. Choi et. al., in “Androgen Controls Apoptosis and Proliferation Via Androgen Receptors in the Adult Rat Penis,” concludes that the “androgen effect is controlled by the expressions of androgen receptors (in the penis)” (163[377]). In a second report by Choi, et. al., in the same journal (report 870), Choi concludes that “the nitric oxide pathway displays affection with androgens.” Guilianao, et. al., in report 858 of the same journal entitled “Comparative Study of Blood Flow, Oxygen Tension (pO₂) and Temperature Changes in the Corpus Cavernaosa of the Rat Penis and the Vagina During Electrically Induced Sexual Responses in Rats” conclude that “The vascular component of sexual responses were comparable in males and females.” Since the clitoris and the penis are analogous structures, “peripheral libido” can be assumed to increase the potential activity of the nitric oxide synthase pathway relative to the testosterone milieu and present in the female clitoris. The testosterone actively in peripheral libido is imparted by the androgen receptors in the clitoral tissues.

These recently reported studies give a receptor/cellular mechanism of physiology to explain peripheral libido. This gives a better understanding of Dr. Kaplan's observations in “The Female Androgen Deficiency Syndrome”: before testosterone treatment, patients confirmed the “deadness of their clitoris,” and, after testosterone, their reported a return of clitoral sensitivity and orgasm capacity. (It is noted that all of the above-cited references are incorporated herein by reference in their entirety).

Thus, increasing the testosterone level in females by such above-recited methodology in conjunction with a topical application of a sensitizing cream as described in my aforementioned U.S. patent application Ser. No. 09/469,959, filed Dec. 22, 1999, and which is incorporated herein by reference in its entirety, comprises the significant improvement in clitoral sensitization and stimulation and accomplishes the objects of the present invention.

The invention thus comprises an arrangement for the treatment of clitoral dysfunction of a female comprising: an augmentation of testosterone for the female to supplement testosterone levels and improve the libido of the female and a compound of menthol and L-Arginine for application onto the clitoris of the female. The augmentation of testosterone may comprise an injection of Testosterone ethanate. The augmentation of testosterone may also comprise a sublingual application of S-Methyltestosterone. The augmentation of testosterone may also comprise a transdermal application of testosterone. The augmentation of testosterone may also comprise an application of a testosterone precursor to the female. The testosterone precursor may comprise DHEA or DHEA-S. The augmentation of testosterone may comprise displacement of testosterone from any circulating loosely-bound testosterone by Angelica sinensis. The augmentation of testosterone may comprise displacement of testosterone from any circulating loosely-bound testosterone by Withania somniferum. The augmentation of testosterone may comprise displacement of testosterone from any circulating loosely-bound testosterone by Tarnera diffusa.

The invention also includes a method of treatment of clitoral dysfunction of a female comprising the steps of: providing an augmentation of testosterone for the female to supplement testosterone levels and improve the libido of the female; and applying a compound of menthol and L-Arginine to the clitoris of the female. The method of augmentation of testosterone may comprise an injection of Testosterone ethanate. The method of augmentation of testosterone may comprise a sublingual application of S-Methyltestosterone. The method of augmentation of testosterone may comprise a transdermal application of testosterone. The method of augmentation of testosterone may comprise an application of a testosterone precursor to the female. The testosterone precursor may comprise DHEA. The testosterone may also comprise DHEA-S. The method of treatment of clitoral dysfunction of a female may also include the step of: displacing testosterone from any circulating loosely-bound testosterone by Angelica sinensis to supplement and elevate the circulating free testosterone in the female or the step of displacing testosterone from any circulating loosely-bound testosterone by Withania somniferum to supplement and elevate the circulating free testosterone or the step of displacing of testosterone from any circulating loosely-bound testosterone by Tarnera diffusa to supplement and elevate the circulating free testosterone in the female

1. An arrangement for the treatment of clitoral dysfunction of a female, so as to decrease the arousal time of that female from the time of initiation of foreplay to the time of her complete clitoral erection, said arrangement comprising:
an augmentation of testosterone for said female to supplement testosterone levels and improve the libido of said female; and
a compound of menthol and L-Arginine for application to the clitoris of said female.

2. The arrangement for the treatment of clitoral dysfunction of a female as recited in claim 1, wherein said augmentation of testosterone comprises an injection of Testosterone ethanate.

3. The arrangement for the treatment of clitoral dysfunction of a female as recited in claim 1, wherein said augmentation of testosterone comprises a sublingual application of 5-Methyltestosterone.

4. The arrangement for the treatment of clitoral dysfunction of a female as recited in claim 1, wherein said augmentation of testosterone comprises a transdermal application of testosterone.

5. The arrangement for the treatment of clitoral dysfunction of a female as recited in claim 1, wherein said augmentation of testosterone comprises an application of a testosterone precursor to said female.

6. The arrangement for the treatment of clitoral dysfunction of a female as recited in claim 5, wherein said testosterone precursor comprises DHEA.

7. The arrangement for the treatment of clitoral dysfunction of a female as recited in claim 5, wherein said testosterone precursor comprises DHEA-S.

8. The arrangement for the treatment of clitoral dysfunction of a female as recited in claim 1, wherein said augmentation of testosterone comprises displacement of testosterone from any circulating loosely-bound testosterone by Angelica sinensis.

9. The arrangement for the treatment of clitoral dysfunction of a female as recited in claim 1, wherein said augmentation of testosterone comprises displacement of testosterone from any circulating loosely-bound testosterone by Withania somniferum.

10. The arrangement for the treatment of clitoral dysfunction of a female as recited in claim 1, wherein said augmentation of testosterone comprises displacement of testosterone from any circulating loosely-bound testosterone by Tarnera diffusa.

11. A method of treatment of clitoral dysfunction of a female so as to decrease the arousal time of that female from the time of initiation of foreplay to the time of her complete clitoral erection, said arrangement comprising the steps of:
providing an augmentation of testosterone for said female to supplement testosterone levels and improve the libido of said female; and
applying a compound of menthol and L-Arginine to the clitoris of said female.

12. The method of treatment of clitoral dysfunction of a female as recited in claim 11, wherein said augmentation of testosterone comprises an injection of Testosterone ethanate.

13. The method of treatment of clitoral dysfunction of a female as recited in claim 11, wherein said augmentation of testosterone comprises a sublingual application of 5-Methyltestosterone.

14. The method of treatment of clitoral dysfunction of a female as recited in claim 11, wherein said augmentation of testosterone comprises a transdermal application of testosterone.

15. The method of treatment of clitoral dysfunction of a female as recited in claim 11, wherein said augmentation of testosterone comprises an application of a testosterone precursor to said female.

16. The method of treatment of clitoral dysfunction of a female as recited in claim 15, wherein said testosterone precursor comprises DHEA.

17. The method of treatment of clitoral dysfunction of a female as recited in claim 15, wherein said testosterone precursor comprises DHEA-S.

18. The method of treatment of clitoral dysfunction of a female as recited in claim 11, including the step of:
displacing testosterone from any circulating loosely-bound testosterone by Angelica sinensis to supplement and elevate the circulating free testosterone.

19. The method of treatment of clitoral dysfunction of a female as recited in claim 11, including the step of:
displacing testosterone from any circulating loosely-bound testosterone by Withania somniferum to supplement and elevate the circulating free testosterone.

20. The method of treatment of clitoral dysfunction of a female as recited in claim 11, including the step of:
displacing of testosterone from any circulating loosely-bound testosterone by Tarnera diffusa to supplement and elevate the circulating free testosterone.

21. An arrangement for the treatment of clitoral dysfunction of a female so as to increase the intensity of that female's orgasm, said arrangement comprising:
an augmentation of testosterone for said female to supplement testosterone levels and improve the libido of said female; and
a compound of menthol and L-Arginine for application to the clitoris of said female.

22. The arrangement for the treatment of clitoral dysfunction of a female as recited in claim 21, wherein said augmentation of testosterone comprises an injection of Testosterone ethanate.

23. The arrangement for the treatment of clitoral dysfunction of a female as recited in claim 21, wherein said augmentation of testosterone comprises a sublingual application of 5-Methyltestosterone.

24. The arrangement for the treatment of clitoral dysfunction of a female as recited in claim 21, wherein said augmentation of testosterone comprises a transdermal application of testosterone.

25. The arrangement for the treatment of clitoral dysfunction of a female as recited in claim 21, wherein said augmentation of testosterone comprises an application of a testosterone precursor to said female.

26. The arrangement for the treatment of clitoral dysfunction of a female as recited in claim 25, wherein said testosterone precursor comprises DHEA.

27. The arrangement for the treatment of clitoral dysfunction of a female as recited in claim 25, wherein said testosterone comprises DHEA-S.

28. The arrangement for the treatment of clitoral dysfunction of a female as recited in claim 21, wherein said augmentation of testosterone comprises displacement of testosterone from any circulating loosely-bound testosterone by Angelica sinensis.

29. The arrangement for the treatment of clitoral dysfunction of a female as recited in claim 21, wherein said augmentation of testosterone comprises displacement of testosterone from any circulating loosely-bound testosterone by Withania somniferum.

30. The arrangement for the treatment of clitoral dysfunction of a female as recited in claim 21, wherein said augmentation of testosterone comprises displacement of testosterone from any circulating loosely-bound testosterone by Tarnera diffusa.

31. A method of treatment of clitoral dysfunction of a female so as to increase the intensity of that female's orgasm comprising the steps of:
providing an augmentation of testosterone for said female to supplement testosterone levels and improve the libido of said female; and
applying a compound of menthol and L-Arginine to the clitoris of said female.

32. The method of treatment of clitoral dysfunction of a female as recited in claim 31, wherein said augmentation of testosterone comprises an injection of Testosterone ethanate.

33. The method of treatment of clitoral dysfunction of a female as recited in claim 31, wherein said augmentation of testosterone comprises a sublingual application of 5-Methyltestosterone.

34. The method of treatment of clitoral dysfunction of a female as recited in claim 31, wherein said augmentation of testosterone comprises a transdermal application of testosterone.

35. The method of treatment of clitoral dysfunction of a female as recited in claim 31, wherein said augmentation of testosterone comprises an application of a testosterone precursor to said female.

36. The method of treatment of clitoral dysfunction of a female as recited in claim 35, wherein said testosterone precursor comprises DHEA.

37. The method of treatment of clitoral dysfunction of a female as recited in claim 35, wherein said testosterone precursor comprises DHEA-S.

38. The method of treatment of clitoral dysfunction of a female as recited in claim 31, including the step of:
displacing testosterone from any circulating loosely-bound testosterone by Angelica sinensis to supplement and elevate the circulating free testosterone.

39. The method of treatment of clitoral dysfunction of a female as recited in claim 31, including the step of:
displacing testosterone from any circulating loosely-bound testosterone by Withania somniferum to supplement and elevate the circulating free testosterone.

40. The method of treatment of clitoral dysfunction of a female as recited in claim 31, including the step of:
displacing of testosterone from any circulating loosely-bound testosterone by Tarnera diffusa to supplement and elevate the circulating free testosterone.

41. An arrangement for the treatment of clitoral dysfunction of a female so as to increase that female's number of multiple orgasms, said arrangement comprising:
an augmentation of testosterone for said female to supplement testosterone levels and improve the libido of said female; and
a compound of menthol and L-Arginine for application to the clitoris of said female.

42. The arrangement for the treatment of clitoral dysfunction of a female as recited in claim 41, wherein said augmentation of testosterone comprises an injection of Testosterone ethanate.

43. The arrangement for the treatment of clitoral dysfunction of a female as recited in claim 41, wherein said augmentation of testosterone comprises a sublingual application of 5-Methyltestosterone.

44. The arrangement for the treatment of clitoral dysfunction of a female as recited in claim 41, wherein said augmentation of testosterone comprises a transdermal application of testosterone.

45. The arrangement for the treatment of clitoral dysfunction of a female as recited in claim 41, wherein said augmentation of testosterone comprises an application of a testosterone precursor to said female.

46. The arrangement for the treatment of clitoral dysfunction of a female as recited in claim 45, wherein said testosterone precursor comprises DHEA.

47. The arrangement for the treatment of clitoral dysfunction of a female as recited in claim 45, wherein said testosterone precursor comprises DHEA-S.

48. The arrangement for the treatment of clitoral dysfunction of a female as recited in claim 41, wherein said augmentation of testosterone comprises displacement of testosterone from any circulating loosely-bound testosterone by Angelica sinensis.

49. The arrangement for the treatment of clitoral dysfunction of a female as recited in claim 41, wherein said augmentation of testosterone comprises displacement of testosterone from any circulating loosely-bound testosterone by Withania somniferum.

50. The arrangement for the treatment of clitoral dysfunction of a female as recited in claim 41, wherein said augmentation of testosterone comprises displacement of testosterone from any circulating loosely-bound testosterone by Tarnera diffusa.

51. A method of treatment of clitoral dysfunction of a female so as to increase that female's number of multiple orgasms, the method comprising the steps of:
providing an augmentation of testosterone for said female to supplement testosterone levels and improve the libido of said female; and
applying a compound of menthol and L-Arginine to the clitoris of said female.

52. The method of treatment of clitoral dysfunction of a female as recited in claim 51, wherein said augmentation of testosterone comprises an injection of Testosterone ethanate.

53. The method of treatment of clitoral dysfunction of a female as recited in claim 51, wherein said augmentation of testosterone comprises a sublingual application of 5-Methyltestosterone.

54. The method of treatment of clitoral dysfunction of a female as recited in claim 51, wherein said augmentation of testosterone comprises a transdermal application of testosterone.

55. The method of treatment of clitoral dysfunction of a female as recited in claim 51, wherein said augmentation of testosterone comprises an application of a testosterone precursor to said female.

56. The method of treatment of clitoral dysfunction of a female as recited in claim 55, wherein said testosterone precursor comprises DHEA.

57. The method of treatment of clitoral dysfunction of a female as recited in claim 55, wherein said testosterone precursor comprises DHEA-S.

58. The method of treatment of clitoral dysfunction of a female as recited in claim 51, including the step of:
displacing testosterone from any circulating loosely-bound testosterone by Angelica sinensis to supplement and elevate the circulating free testosterone.

59. The method of treatment of clitoral dysfunction of a female as recited in claim 51, including the step of:
displacing testosterone from any circulating loosely-bound testosterone by Withania somniferum to supplement and elevate the circulating free testosterone.

60. The method of treatment of clitoral dysfunction of a female as recited in claim 51, including the step of:
displacing of testosterone from any circulating loosely-bound testosterone by Tarnera diffusa to supplement and elevate the circulating free testosterone.
